(19) United States
(12) Patent Application Publication
(10) Pub. No.: US 2023/0302076 A1
(43) Pub. Date: Sep. 28, 2023

(54) Natural and Organic Composition for Preventing Hair Loss and Promoting Hair Growth

(71) Applicant: Nina Street, Winston Salem, NC (US)

(72) Inventor: Nina Street, Winston Salem, NC (US)

(21) Appl. No.: 18/053,423

(22) Filed: Nov. 8, 2022

Related U.S. Application Data
(60) Provisional application No. 63/322,814, filed on Mar. 23, 2022.

(51) Int. Cl. A61K 36/53; A61K 36/889; A61K 36/63; A61K 36/736; A61K 36/47; A61K 36/87; A61K 36/48; A61K 36/54; A61K 36/61; A61K 36/886; A61K 36/00; A61K 36/28; A61K 36/55; A61K 36/185; A61K 36/23; A61K 31/355

(52) U.S. Cl. CPC A61K 36/53; A61K 36/889; A61K 36/63; A61K 36/736; A61K 36/47; A61K 36/87; A61K 36/48; A61K 36/54; A61K 36/61; A61K 36/886; A61K 36/00; A61K 36/28; A61K 36/55; A61K 36/185; A61K 36/23; A61K 31/355

(57) ABSTRACT

The present invention relates to a composition to help facilitate hair growth while moisturizing the scalp. The composition contains natural and organic ingredients and helps stimulate hair roots to promote growth while relieving itchy and dry skin disorders like alopecia, eczema, and psoriasis. The composition includes olive oil, coconut oil, shea butter, almond, castor oil, grape seed oil, fenugreek oil, aloe vera oil, avocado oil, lavender oil, rosemary oil, tea tree oil, basil, oregano, natural vitamin e, saw palmetto, nettle, horsetail, bhringraj, flax seed, amla, and black seed oil in other embodiments, the composition includes ylang ylang, hemp seed, onion oil, moringa oil, burdock root, andiroba, and honey.

### CROSS-REFERENCE TO RELATED APPLICATION

The present application claims priority to, and the benefit of, U.S. Provisional Application No. 63/322,814, which was filed on Mar. 23, 2022, and is incorporated herein by reference in its entirety.

### FIELD OF THE INVENTION

The present invention relates generally to the field of haircare products. More specifically, the present invention relates to a novel skin and haircare product for inhibiting hair loss and promoting hair growth and healthy-looking skin. The product has a natural and organic composition that can be used for treatment of all types of hair losses and various skin disorders. The composition is used for treating some various types of alopecia without any discomfort and in a cost-effective manner. Accordingly, the present disclosure makes specific reference thereto. Nonetheless, it is to be appreciated that aspects of the present invention are also equally applicable to other like applications, devices, and methods of manufacture.

### BACKGROUND

By way of background, hair loss is a common problem in both males and females. Hair loss can be caused due to hereditary and in males, it is called male pattern hair loss or male pattern baldness. It is most common cause for hair loss and is also known as “androgenic alopecia,” wherein human males begin losing scalp hair at the temples and on the crown of the head in early adult life. Androgenetic alopecia is a genetically predetermined disorder due to an excessive response to androgens. Similar patterned baldness occurs in women, though it progresses more slowly and does not reach the end stage of complete denudation and is known as female pattern hair loss.

Alopecia areata is a disease that develops when a user body's immune system attacks hair follicles, causing hair loss. As a result, hair loss can take place anywhere on the body, including the scalp. Many other reasons such as itchy skin, dandruff, eczema, and more can lead to hair loss in both males and females. As a result of excessive hair loss and baldness due to hair loss, individuals opt for expensive hair regeneration treatments, use of hair wigs, and more. Hair transplantation or regeneration methods include transplantation of plugs of hair-bearing skin from the back of the scalp into the bald areas. The procedure is not only costly but also painful. Surgical treatments available for the restoration of hair, particularly to the scalp are painful, expensive, and suffer from the inherent dangers associated with any invasive surgery. Yet, surgical treatments are rarely applicable to sufferers of alopecia areata in which spontaneous regrowth of hair which subsequently falls out again, often occurs.

Many other approaches for treating hair loss, including alopecia, have been tried. Such approaches include therapies comprising non-specific immunosuppressants such as corticosteroids or UVA treatment, contact dermatitis inducers, specific immunosuppressants, and more. However, none of these has been generally accepted as effective.

For preventing preliminary hair loss, individuals try to keep their scalp moisturized and hair strengthened using hair products. However, conventional hair products have chemical composition which can be detrimental for hair and skin for long-term applications and may cause itchiness and rashes. Further, such hair products are ineffective and are expensive too for consumers.

Alternative treatments such as essential oils and acupuncture and experimental and theoretical treatments such as cytokines, biologicals, desensitization, oral tolerance, and gene therapy have been variously employed in the treatment of alopecia with less success. Individuals desire a novel and non-obvious hair product solution that is safe to use and facilitates hair growth while moisturizing the scalp.

Therefore, there exists a long-felt need in the art for a haircare product designed to help facilitate hair growth while moisturizing the scalp. There is also a long-felt need in the art for a haircare product that stimulates hair roots to promote hair growth. Additionally, there is a long-felt need in the art for a skin and haircare product that does not contain any harmful chemicals. Moreover, there is a long-felt need in the art for a skin and haircare product for providing relief from itchy and dry skin disorders like alopecia, eczema, psoriasis, and more. Further, there is a long-felt need in the art for a haircare product that can be used for long-term application by users. Furthermore, there is a long-felt need in the art for a skin and haircare product that reduces requirement of using expensive and discomforting hair regeneration and transplantation techniques. Finally, there is a long-felt need in the art for a skin and haircare product that offers relief from itchiness and rashes while helping prevent hair loss.

The subject matter disclosed and claimed herein, in one embodiment thereof, comprises a composition for the treatment of hair loss. The composition comprising olive oil, coconut oil, shea butter, almond, castor oil, grape seed oil, fenugreek oil, aloe vera oil, avocado oil, lavender oil, rosemary oil, tea tree oil, basil, oregano, natural vitamin E, saw palmetto, nettle, horsetail, bhringraj, flax seed, amla, and black seed oil.

In this manner, the skin and haircare composition of the present invention accomplishes all of the forgoing objectives and provides users with a skin and haircare product designed to help facilitate hair growth while moisturizing the scalp. The composition helps stimulate hair roots to promote growth while relieving itchy and dry skin disorders like alopecia, eczema, psoriasis, and more. The composition eliminates the use of expensive creams and hair transplant techniques.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a composition for the treatment of hair loss. The composition comprising olive oil, coconut oil, shea butter, almond, castor oil, grape seed oil, fenugreek oil, aloe vera oil, avocado oil, lavender oil, rosemary oil, tea tree oil, basil, oregano, thyme, vitamin E, saw palmetto, nettle, horsetail, bhringraj, flax seed, amla, and black seed oil.

In yet another embodiment, a composition for the treatment of skin disorders, such as eczema, psoriasis, skin rashes, fungus, sunburn, and more is disclosed. The composition comprises shea butter, olive oil, coconut oil, cocoa butter, grape seed oil, avocado oil, water, aloe vera, lavender oil, rosemary oil, tea tree oil, nettle, basil, thyme, burdock root, vitamin E, black seed, andiroba, and honey.

In yet another embodiment, a composition for the treatment of itchy scalp, reducing dandruff and promoting hair growth is disclosed. The composition comprises olive oil, rice bran, flax seed oil, bhringraj, black castor oil, black seed oil, ylang ylang, hemp seed, onion oil, moringa oil, ginger, calendula oil, tea tree, rosemary, clove, saw palmetto, and ginseng oil.

In yet another embodiment, a method of topically applying a skin composition to address fungus beneath the fingernails and/or toe nails is described. The method includes the steps of topically applying an effective amount of the composition comprising olive oil, lavender, rosemary, tea tree, basil, thyme, burdock root, oregano, and vitamin E.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of a container storing a haircare composition of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a haircare product designed to help facilitate hair growth while moisturizing the scalp. There is also a long-felt need in the art for a haircare product that stimulates hair roots to promote hair growth. Additionally, there is a long-felt need in the art for a skin and haircare product that does not contain any harmful chemicals. Moreover, there is a long-felt need in the art for a skin and haircare product for providing relief from itchy and dry skin disorders like alopecia, eczema, psoriasis, and more. Further, there is a long-felt need in the art for a haircare product that can be used in long-term or multiple applications by users. Furthermore, there is a long-felt need in the art for a skin and haircare product that reduces requirement of using expensive and discomforting hair regeneration and transplantation techniques. Finally, there is a long-felt need in the art for a skin and haircare product that offers relief from itchiness and rashes while helping prevent hair loss.

The present invention, in one exemplary embodiment, is a method of topically applying a skin and haircare composition on follicles for preventing hair loss and promoting hair growth. The method includes the steps of topically applying an effective amount of the composition comprising olive oil, coconut oil, shea butter, almond, castor oil, grape seed oil, fenugreek oil, aloe vera oil, avocado oil, lavender oil, rosemary oil, tea tree oil, basil, oregano, natural vitamin E, saw palmetto, nettle, horsetail, bhringraj, flax seed, amla, and black seed oil to a subject in such need.

The skin and haircare product composition preferably comprises natural and organic ingredients for the treatment and prevention of hair loss. The ingredients are extracted from natural sources and are preferably purified before inclusion in the composition in order that the composition is substantially in pure form. The composition is ideal for use in the treatment of alopecia such as alopecia areata and alopecia androgenetic. It should be appreciated that the haircare composition of various embodiments of the present invention can be used for treatment and prevention of alopecia of various types.

Olive oil is rich in fatty acids, omega-3, vitamin E, and antioxidants and has a chemical composition obtained naturally by pressing olives. Olive oil strengthens hair follicles, has a soothing impact on the scalp and even stimulates hair growth. Further, olive oil acts as a great moisturizer and prevents dryness as olive oil has antibacterial and anti-fungal properties and contains squalene and oleic acid. Olive oil's moisturizing effect helps reduce itchiness, deals with the dryness, fights dandruff, and unclogs the clogged follicles. Olive oil blocks the production of dihydrotestosterone, a hormone that causes hair loss.

Coconut oil helps slow down hair loss by penetrating deep into hair shafts to prevent protein loss, which in turn prevents breakage. Coconut oil simultaneously moisturizes and removes buildup around hair follicles to jumpstart hair growth. More specifically, coconut oil is a medium-chain triglycerides of lauric acid, increasing its affinity for hair proteins and allowing it to travel inside the hair shaft more than other commonly used oils, thereby preventing hair loss.

Shea butter (vitellaria paradoxa) is a refined triglyceride fat from the shea tree, is gentle, has moisturizing and gentle anti-inflammatory properties. Shea butter contains a range of nutrients including ingredients such as oleic acid, stearic acid, palmitic acid, and linoleic acid. These ingredients provide nutrients to scalp and follicles, conditioning them and providing them with stimulation that help condition and smooth hair. Additionally, vitamins A, E, and F, promote circulation and healthy skin cell growth.

Zinc deficiency generally leads to alopecia and thinning of hair. Almond oil has magnesium, calcium and zinc that are proven to promote hair growth and protect against hair loss. Almond oil contains vitamin E and biotin, which helps repair damaged hair. Further, almond oil is rich in protein and Omega-3 fatty acids, that makes hair free of frizz and lustrous.

PGD₂ is important for development of the male reproductive system. PDG₂ production is fueled by testosterone through a process known as the arachidonic acid cascade, however, overproduction of PGD₂ can slow hair growth. Ricinoleic acid, a major component of castor oil, functions as a potentially effective inhibitor of PDG₂. Direct application of castor oil to the scalp is an effective way to boost hair growth and improve the overall quality of scalp and hair.

Proanthocyanidins present in grape seeds oil function as agents inducing hair growth. Proanthocyanidins converts hair-cycle activity from the telogen phase to the anagen phase and thus help in hair regrowth. Grapeseed oil contains vitamin E, flavonoids, linoleic acid, and OPCs, all of which are necessary for healthy hair growth and vitality.

Fenugreek (trigonella foenum-graecum) slows down the ability of DHT (dihydrotestosterone) to attach to hair follicles. DHT when attached to hair follicles, sooner or later, causes hair loss. Anti-inflammatory properties of fenugreek also help in healing of follicles and combating the effects of androgenetic alopecia.

Aloe vera includes vitamins A, C, E, B12, and choline and application of aloe vera to follicles helps in hair growth and improved hair health. Aloe vera also helps in improving blood circulation and nutrition to the hair follicles in addition to providing anti-bacterial property and anti-inflammatory property. Aloe vera also acts as a biological vehicle to aid penetration and absorption of other bio-active ingredient into skin and follicles.

Avocado oil includes potassium and magnesium that help seal cuticle cells, which prevents hair from breaking. Avocado oil contains a plurality of fatty acids including palmitoleic acid, linoleic acid, palmitic acid, and oleic acid for providing strength to hair and moisturizing, nourishing the follicles.

Essential oils such as lavender, rosemary, tea tree, basil, and oregano help in aromatherapy and have hair growth-promoting properties. Such oils have pharmacologically active stimulants and can be therapeutic in patients with alopecia. More specifically, rosemary reduces the dihydrotestosterone levels in hair follicles, therefore, helps prevent hair loss and thinning hair. Lavender oil increases blood circulation in the scalp and capable of increasing the number and the depth of hair follicles. Similarly, other oils increase antibacterial activity, optimize hair growth, cleanse and revitalize hair follicles.

Saw palmetto (SP) is a botanical extract that has antiandrogenic properties and helps in hair regrowth. More specifically, saw palmetto is an extract from the berries of the palm tree saw palmetto and decreases dihydrotestosterone (DHT) uptake by hair follicle and decreases the binding of DHT to androgenetic receptors. Saw palmetto helps block the activity of 5-alpha reductase, an enzyme that converts testosterone into DHT. In addition to preserving levels of testosterone, saw palmetto may reduce levels of DHT, which may be involved in hair loss.

Stinging nettle, or Urtica dioica, naturally increases dermal papilla cell growth which are part of the hair follicle and regulate hair growth. Stinging nettle root also has the ability to block DHT and can block the production of DHT by reducing the production of the 5-alpha-reductase enzyme. Nettle leaves are also rich in silica and sulfur, minimizing hair breakage and thinning while also regenerating hair follicles.

Horsetail extract improves the circulation of blood, which in turn leads to healthy hair follicles. Further, it has antioxidant properties that also work as a detox for hair and body and helps with hair growth and stimulating the hair follicles.

Bhringraj (eclipta prostrata) oil increases blood circulation to the scalp and roots, which promotes hair growth. The oil activates the hair follicles, which enhances hair growth. The oil is analgesic, antihaemorrhagic, immunomodulatory and has potent antihepatotoxic activity. Bhringraj is a potent hair vitalizer and has a host of bioactive constituents including flavonoids and phytochemicals like ecliptic, wedelolactone, coumestans, polypeptides, polyacetylenes, thiophene derivatives, steroids, sterols like stigmasterol, heptacosanole, hentriacontanol, and triterpenes.

Flaxseeds contain vitamin E, which diminishes the influence of free radicals on scalp and improves hair growth and stronger hair follicles. Flaxseeds help soothe the scalp as the omega-3 fatty acids help reduce scalp inflammation. Flaxseeds also contain vitamin B and are rich in biotin, riboflavin, niacin, pyridoxine, pantothenic acid, and folic acid, which are responsible for increasing hair growth making hair stronger. Further, alpha-linolenic acid found in flaxseed DHT blockers helps block the enzyme that creates DHT in the body. By preventing DHT from shrinking hair follicles, flaxseed helps prevent hair loss.

Amla oil is a natural oil used for hair health. The oil is made by drying phyllanthus emblica and soaking in a base oil. The oil is then filtered and purified. Amla oil stimulates hair growth, as well as prevents hair loss and early graying. Amla also contains tannins like ellagic acid, chebulagic acid, and pyrogallol, and flavonoids like quercetin that are potent DHT blockers.

The black seeds from nigella sativa are pressed for black seed oil and contains thymoquinone, that has antioxidant properties to help relieve inflammation. Black seed oil helps maintain scalp health by addressing issues such as dandruff while keeping the scalp moisturized.

Andiroba oil has essential fatty acids (EFAs) that promotes healthier hair growth, treating damaged or curly hair without leaving a greasy feeling behind. Andiroba oil stimulates better blood flow, which is a strong factor in growing healthier hair making hair more resilient to breakage, split ends, and dehydration.

Ylang ylang essential oil (and other essential oils) is an effective natural treatment to reduce hair loss caused by stress and alopecia. Ylang ylang essential oil is extracted from the flowers of Cananga odorata (ylang ylang) by steam distillation. It has natural hair growth-boosting and conditioning properties.

Preferably, the skin and haircare composition of the present invention is suitable for topical administration. The compositions of various embodiments contemplated by this invention include compositions adopted for topical application to the human scalp and/or skin. Conventional composition forms for this purpose may include ointments, lotions, pastes, jellies, gels, mousses, powders, skin cream, oil serum, cuticle cleanser and similar known composition forms. To form a cream or ointment a paraffinic fraction and an emulsion base may be used.

Suitable fragrances for use in the composition may include menthol, benzyl alcohol, phenoxyethanol, isopropyl palmitate, isopropyl myristate, phenylethyl salicylate, thymol, isoamyl salicylate, triton X-100 surfactant, and more.

The composition 100 of the present invention will now be described by way of illustration only in the following examples:

Example 1

| | | | | Composition |
|---|---|---|---|---|
| | | | Volume | in percentage |
| | | | in | by weight |
| Ingredients | Volume | | milliliter | (% v/v) |
| Olive Oil | 1.5 | c | 354 | 28.89489 |
| Coconut Oil | 1.25 | c | 295 | 24.07908 |
| Shea Butter | 16 | oz. | 473.1 | 38.60815 |
| Mixture of Almond, | 3 | tbsp. | 44 | 3.591456 |
| Castor Oil, Grape Seed | | | | |
| Oil, Fenugreek Oil, | | | | |
| Aloe Vera Oil, | | | | |
| Avocado Oil | | | | |
| Essential oils | 2 | tsp. | 29.57 | 2.413621 |
| (including Lavender, | | | | |
| Rosemary, Tea Tree, | | | | |
| Basil, Oregano, | | | | |
| Thyme, Oils and | | | | |
| Vitamin E) | | | | |
| Mixture of Saw | 1 | tsp. | 14.78 | 1.206403 |
| Palmetto, Nettle, | | | | |
| Horsetail | | | | |
| Mixture of Bhringraj, | 1 | tbsp. | 14.78 | 1.206403 |

Flax Seed, Amla, Black
Seed Oils

Example 2

| | | | | Composition |
|---|---|---|---|---|
| | | | Volume | in percentage |
| | | | in | by weight |
| Ingredients | Volume | | milliliter | (% v/v) |
| Shea Butter | 16 | oz. | 473.1 | 40.28 |
| Olive Oil | 1 | c | 236.59 | 20.15 |
| Coconut oil | ½ | c | 118.29 | 10.07 |
| Cocoa Butter | 3 | oz. | 85.23 | 7.26 |
| Grape seed oil | ¼ | c | 59.14 | 5.04 |
| Avocado oil | ¼ | c | 59.14 | 5.04 |
| Water | ¼ | c | 59.14 | 5.04 |
| Aloe vera | 3 | tbsp. | 44.36 | 3.78 |
| Mixture (Lavender, | 2 | tsp. | 9.85 | 0.84 |
| Rosemary, Tea Tree, | | | | |
| Nettle, Basil, Thyme, | | | | |
| Burdock Root, | | | | |
| Vitamin E) | | | | |
| Mixture (Black Seed, | 2 | tbsp | 29.57 | 2.52 |

Andiroba, Honey)

Example 3

| | | | | Composition |
|---|---|---|---|---|
| | | | Volume | in percentage |
| | | | in | by weight |
| Ingredients | Volume | | milliliter | (% v/v) |
| Olive Oil | 1 | c | 236.59 | 88.89 |
| Mixture (Lavender, | 2 | tsp. | 29.57 | 11.10 |

Rosemary, Tea Tree,

Basil, Thyme,
Oregano, Vitamin E)

Example 4

| | | | | Composition |
|---|---|---|---|---|
| | | | Volume | in percentage |
| | | | in | by weight |
| Ingredients | Volume | | milliliter | (% v/v) |
| Olive Oil | 1 | c | 236.59 | 72.73 |
| Rice Bran, Flax Seed | 3 | tbsp. | 44.36 | 13.64 |
| oil | | | | |
| Bhringraj, Black Castor | 2 | tbsp. | 29.57 | 9.09 |
| Oil, Black Seed Oil | | | | |
| Ylang Ylang, Hemp | 2 | tsp. | 9.85 | 3.03 |
| Seed, Onion Oil, | | | | |
| Moringa Oil | | | | |
| Ginger, Calendula Oil, | 1 | tsp. | 4.93 | 1.52 |

Tea Tree, Rosemary,
Clove, Saw Palmetto,
Ginseng Oil

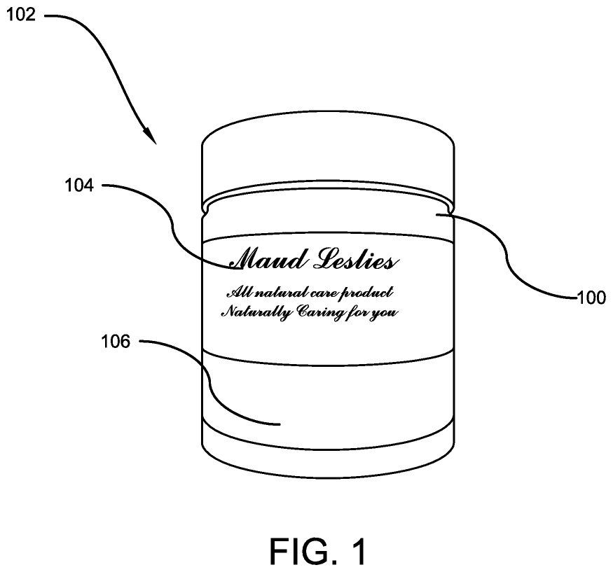

FIG. 1 illustrates a perspective view of a container storing a haircare composition of the present invention in accordance with the disclosed architecture. As illustrated, the exemplary container 102 stores the composition 100 of the present invention in a cream or ointment form. The container 102 has a custom logo 104 that can also display the composition and instructions 106 for using the composition 100.

Certain terms are used throughout the following description and claims to refer to particular features or components. As one skilled in the art will appreciate, different persons may refer to the same feature or component by different names. This document does not intend to distinguish between components or features that differ in name but not composition or function. As used herein “skin and haircare product composition”, “haircare product composition”, and “composition” are interchangeable and refer to the skin and haircare product composition 100 of the present invention.

Notwithstanding the forgoing, the skin and haircare product composition 100 of the present invention can be of any suitable composition, provided that it accomplishes the above stated objectives. One of ordinary skill in the art will appreciate that the skin and haircare product composition 100 as described is for illustrative purposes only, and that many other ingredients and compositions are well within the scope of the present disclosure.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A method for the treatment or management of hair loss, comprising administering to a subject experiencing hair loss a therapeutically effective amount of a composition comprising:
an olive oil;
a coconut oil;
a shea butter;
a first mixture selected from a group consisting of one or more of an almond oil, a castor oil, a grape seed oil, a fenugreek oil, an aloe vera oil, and an avocado oil;
an essential oil selected from a group consisting of a lavender oil, a rosemary oil, a tea tree oil, a basil oil, an oregano oil, and a Vitamin E oil;
a second mixture selected from a group consisting of one or more of a saw palmetto, a nettle leaf, and a horsetail extract; and
a third mixture selected from a group consisting of one or more of a bhringraj oil, a flax seed oil, an amla oil, and a black seed oil.

2. The method of claim 1, wherein said olive oil comprises generally about 28.9% by weight of said composition.

3. The method of claim 1, wherein said coconut oil comprises generally about 24.8% by weight of said composition.

4. The method of claim 1, wherein said shea butter comprises generally about 38.6% by weight of said composition.

5. The method of claim 1, wherein said first mixture comprises generally about 3.6% by weight of said composition.

6. The method of claim 1, wherein said essential oil comprises generally about 2.4% by weight of said composition.

7. The method of claim 1, wherein said second mixture comprises generally about 1.2% by weight of said composition.

8. The method of claim 1, wherein said third mixture comprises generally about 1.2% by weight of said composition.

9. A method for the treatment or management of skin disorders, comprising administering to a subject experiencing skin disorders a therapeutically effective amount of a composition comprising:
an olive oil;
a coconut oil;
a shea butter;
a cocoa butter;
a grape seed oil;
an avocado oil;
a water;
an aloe vera;
a first mixture selected from a group consisting of one or more of a lavender oil, a rosemary oil, a tea tree oil, a nettle leaf, a basil oil, a thyme, a burdock root extract, and a vitamin E oil; and
a second mixture selected from a group consisting of one or more of a black seed oil, an andiroba, and a honey.

10. The method of claim 9, wherein said olive oil comprises generally about 20.1% by weight of said composition.

11. The method of claim 9, wherein said coconut oil comprises generally about 10.1% by weight of said composition.

12. The method of claim 9, wherein said shea butter comprises generally about 40.3% by weight of said composition.

13. The method of claim 9, wherein said first mixture comprises generally about 0.8% by weight of said composition.

14. The method of claim 9, wherein said cocoa butter comprises generally about 7.3% by weight of said composition.

15. The method of claim 9, wherein said second mixture comprises generally about 2.5% by weight of said composition.

16. The method of claim 9, wherein said grape seed oil comprises generally about 5.0% by weight of said composition.

17. The method of claim 9, wherein said avocado oil comprises generally about 5.0% by weight of said composition.

18. The method of claim 9, wherein said water comprises generally about 5.0% by weight of said composition.

19. The method of claim 9, wherein said aloe vera comprises generally about 3.8% by weight of said composition.

20. A method for the treatment or management of fungi and rashes, comprising administering to a subject experiencing skin irritations a therapeutically effective amount of a composition comprising:
an olive oil;
wherein said olive oil comprises generally from about 72% to about 89% by weight of said composition;
a first mixture selected from a group consisting of one or more of a lavender oil, a rosemary oil, a tea tree oil, a basil oil, a thyme oil, an oregano oil, and a vitamin E oil; and
further wherein said first mixture comprises generally from about 11% to about 28% by weight of said composition.
